(19) United States
(12) Patent Application Publication
(10) Pub. No.: US 2022/0310223 A1
(43) Pub. Date: Sep. 29, 2022

(54) Methods, Systems, And Apparatuses For Prescription Processing

(71) Applicant: McKesson Corporation, San Francisco, CA (US)

(72) Inventors: Michael Brett Cohn, Dublin, OH (US); Barrett James Klaas, Pittsburgh, PA (US)

(21) Appl. No.: 17/497,591

(22) Filed: Oct. 8, 2021

Related U.S. Application Data
(60) Provisional application No. 63/164,729, filed on Mar. 23, 2021.

(51) Int. Cl. G16H 20/10

(52) U.S. Cl. CPC G16H 20/10

(57) ABSTRACT

Technologies are provided for processing a prescription supplied by a non-dispensing pharmacy and causing delivery of a medication associated with the prescription. At least some embodiments provide full-stack front-end and back-end prescription processing and home delivery by means of a dispensing pharmacy platform and a central fill platform that provides central fill (CF) functions as a service. The dispensing pharmacy platform can serve as an intermediary between a cohort system that supplies a prescription, and a corresponding non-dispensing pharmacy, and the CF platform.

### CROSS-REFERENCE TO RELATED APPLICATION

This application claims the benefit of and priority to U.S. Provisional Application No. 63/164,729, filed Mar. 23, 2021, the entire contents of which application are hereby incorporated herein by reference.

### SUMMARY

It is to be understood that both the following general description and the following detailed description are illustrative and explanatory only and are not restrictive.

In one embodiment, the disclosure provides a computer-implemented method for prescription processing. The computer-implemented method includes receiving, by a dispensing pharmacy system, a prescription fill request. The dispensing pharmacy system may receive the prescription fill request from a first non-dispensing pharmacy system. The prescription fill request may comprise a prescription identifier associated with a patient and at least one medication, a first identifier associated with the first non-dispensing pharmacy system, and a second identifier associated with the first non-dispensing pharmacy system. The computer-implemented method also includes generating, based on the prescription fill request, an order message associated with the prescription identifier, where a first portion of the order message comprises the first identifier and the second identifier; sending, to a central fill system, the order message, where the order message causes the central fill system to dispense the at least one medication; determining, based on an indication from the central fill system, that the prescription fill request has been processed, where the indication identifies the order message; determining, based on the prescription fill request having been processed, and based on the first portion of the order message, a fulfillment notification indicating the prescription fill request has been processed; and sending, based on the fulfillment notification, a message indicating that the at least one medication has been dispensed.

In another embodiment, the disclosure provides an apparatus for prescription processing. The apparatus includes one or more processors; and one or more memory devices storing computer-executable instructions that, when executed by the one or more processors, cause the apparatus to receive, from a first non-dispensing pharmacy system a prescription fill request. The prescription fill request comprises a prescription identifier associated with a patient and at least one medication, a first identifier associated with the first non-dispensing pharmacy system, and a second identifier associated with the first non-dispensing pharmacy system. The computer-executable instructions, when executed by the one or more processors, also cause the apparatus to: generate, based on the prescription fill request, an order message associated with the prescription identifier, where a first portion of the order message comprises the first identifier and the second identifier: send, to a central fill system, the order message, where the order message causes the central fill system to dispense the at least one medication; determine, based on an indication from the central fill system, that the prescription fill request has been processed, where the indication identifies the order message; determine, based on the prescription fill request having been processed, and based on the first portion of the order message, a fulfillment notification indicating the prescription fill request has been processed; and send, based on the fulfillment notification, a message indicating that the at least one medication was dispensed.

In yet another embodiment, the disclosure provides a computer-program product for prescription processing. The computer-program product includes a non-transitory computer-readable storage medium comprising processor-executable instructions that, when executed by one or more processors of a computing device, cause the computing device to receive, from a first non-dispensing pharmacy system, a prescription fill request. The prescription fill request comprises a prescription identifier associated with a patient and at least one medication, a first identifier associated with the first non-dispensing pharmacy system, and a second identifier associated with the first non-dispensing pharmacy system. The processor-executable instructions also are executable by the one or more processors to cause the computing device to: generate, based on the prescription fill request, an order message associated with the prescription identifier, where a first portion of the order message comprises the first identifier and the second identifier; send, to a central fill system, the order message, where the order message causes the central fill system to dispense the at least one medication; determine, based on an indication from the central fill system, that the prescription fill request has been processed, where the indication identifies the order message; determine, based on the prescription fill request having been processed, and based on the first portion of the order message, a fulfillment notification indicating the prescription fill request has been processed; and send, based on the fulfillment notification, a message indicating that the at least one medication was dispensed.

Additional elements or advantages of this disclosure will be set forth in part in the description which follows, and in part will be apparent from the description, or may be learned by practice of the subject disclosure. The advantages of the subject disclosure can be attained by means of the elements and combinations particularly pointed out in the appended claims.

This summary is not intended to identify critical or essential features of the disclosure, but merely to summarize certain features and variations thereof. Other details and features will be described in the sections that follow. Further, both the foregoing general description and the following detailed description are illustrative and explanatory only and are not restrictive of the embodiments of this disclosure.

### BRIEF DESCRIPTION OF THE DRAWINGS

The annexed drawings are an integral part of the disclosure and are incorporated into the subject specification. The drawings illustrate example embodiments of the disclosure and, in conjunction with the description and claims, serve to explain at least in part various principles, elements, or aspects of the disclosure. Embodiments of the disclosure are described more fully below with reference to the annexed drawings. However, various elements of the disclosure can be implemented in many different forms and should not be construed as limited to the implementations set forth herein. Like numbers refer to like elements throughout.

FIG. 1 illustrates an example of an operating environment for virtual pharmacies, in accordance with one or more embodiments of this disclosure.

FIG. 2 illustrates an example of an architecture of the operating environment shown in FIG. 1, in accordance with one or more embodiments of this disclosure.

FIG. 3 illustrates an example of a process flow for fulfillment of a new prescription, in accordance with one or more embodiments of this disclosure.

FIG. 4 illustrates an example of a process for generating a prescription fill request, in accordance with one or more embodiments of this disclosure.

FIG. 5 illustrates an example of a process for prescription fill, in accordance with one or more embodiments of this disclosure.

FIG. 6 illustrates an example of another process flow for fulfillment of a new prescription, in accordance with one or more embodiments of this disclosure.

FIG. 7 illustrates an example of a process flow for fulfillment of a prescription refill, in accordance with one or more embodiments of this disclosure.

FIG. 8 illustrates an example of a process for composing a prescription refill request, in accordance with one or more embodiments of this disclosure.

FIG. 9 illustrates an example of a process for prescription refill, in accordance with one or more embodiments of this disclosure.

FIG. 10 illustrates an example of another process flow for fulfillment of a prescription refill, in accordance with one or more embodiments of this disclosure.

FIG. 11 illustrates an example of a method for filling a prescription, in accordance with one or more embodiments of this disclosure.

FIG. 12 illustrates an example of another operating environment that can implement virtual pharmacies in accordance with one or more embodiments of this disclosure.

### DETAILED DESCRIPTION

The disclosure recognizes and addresses, among other technical challenges, the issue of supplying medications using a non-dispensing pharmacy as a fulfilment pharmacy. To that end, embodiments of this disclosure, individually or in combination, permit processing a prescription supplied by a non-dispensing pharmacy and also causing delivery of a medication associated with the prescription. Embodiments described herein provide full-stack front-end and back-end prescription processing and home delivery by means of a dispensing pharmacy platform and a central fill platform that provides CF functions as a service. The dispensing pharmacy platform can serve as an intermediary between a cohort system that supplies a prescription, and a corresponding non-dispensing pharmacy, and the CF platform.

The embodiments described herein can operate on prescriptions supplied by multiple disparate cohort systems having respective private-labeled non-dispensing pharmacies. By utilizing a custom data field within an order message corresponding to a prescription, embodiments of the disclosure can solve the issue of delineating patients and their prescriptions between cohort systems associated with respective non-dispensing pharmacies. Indeed, that issue can be avoided without convoluted, risky data processing or protected health information (PHI) processing, or both. As a result, embodiments of the disclosure can process large volumes of prescriptions while efficiently using computing resources (such as computing time, storage, and/or network bandwidth, for example). The efficient use of computing resources can mitigate network congestion and/or other types of blockade of access to processing time and/or storage resources.

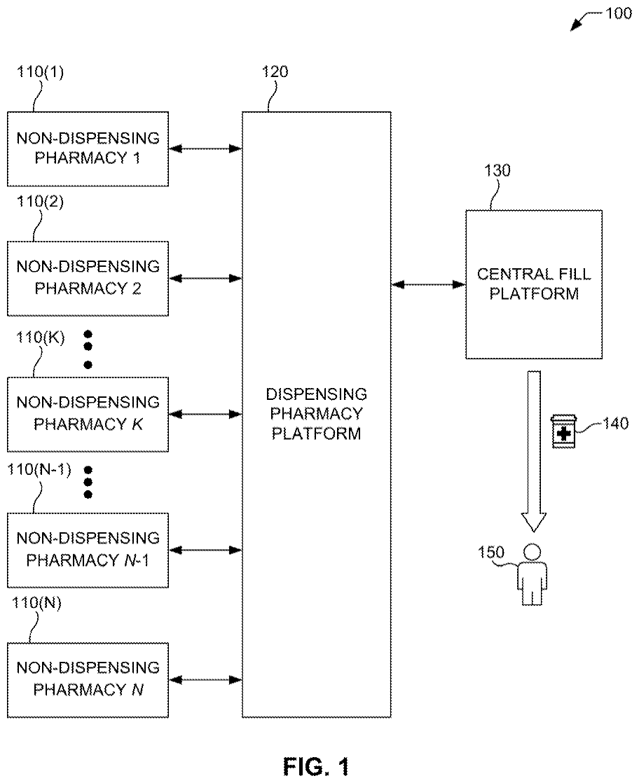

With reference to the drawings, FIG. 1 illustrates an operating environment 100 for virtual pharmacies, in accordance with one or more embodiments of this disclosure. The operating environment 100 comprises a group of non-dispensing pharmacies, including non-dispensing pharmacy 1 110(1), non-dispensing pharmacy 2 110(2), . . . , non-dispensing pharmacy K 110(K), . . . , non-dispensing pharmacy N-1 110(N-1), and non-dispensing pharmacy N 110(N). Although N is depicted as being greater than four, the disclosure is not limited in that respect, and fewer or more than four non-dispensing pharmacies can be contemplated. Each non-dispensing pharmacy in the group of non-dispensing pharmacies can be assigned a respective National Provider Identifier (NPI) number and National Council for Prescription Drug Programs (NCPDP) processor identification number (also referred to as BIN). Additionally, or in some cases, each non-dispensing pharmacy in the group of non-dispensing pharmacies can be assigned a unique fax number. Further each non-dispensing pharmacy in the group of non-dispensing pharmacies can be functionally coupled to one or several remotely located computing devices (not depicted in FIG. 1). Some of the computing devices can permit originating prescriptions or refills of prescriptions. Other ones of the computing devices can administer the operation of a particular non-dispensing pharmacy. The remotely located computing devices corresponding to a non-dispensing pharmacy J 110(J) (J=1, 2, . . . , K . . . N) can constitute a vendor or cohort.

Each non-dispensing pharmacy in the group of non-dispensing pharmacies is functionally coupled to a dispensing pharmacy platform 120. The dispensing pharmacy platform 120 has the licensure to dispense medications, and also has an NPI number and an NCPDP processor identification number (or BIN). The dispensing pharmacy platform 120 can be geographically distributed and includes a network of the retail pharmacies. Each one of those retail pharmacies is licensed to dispense medications. To that end, each one of the retail pharmacies can rely on a central fill (CF) platform 130 that provides CF functions as a service. Those functions include the dispensing and shipping of medications. The CP platform 130 can dispense and ship a medication 140 to a patient 150, where the medication 140 can be prescribed by a source device (not depicted in FIG. 1) corresponding one of the non-dispensing pharmacies 110(1) to 110(N). In FIG. 1, shipping is depicted with a straight arrow.

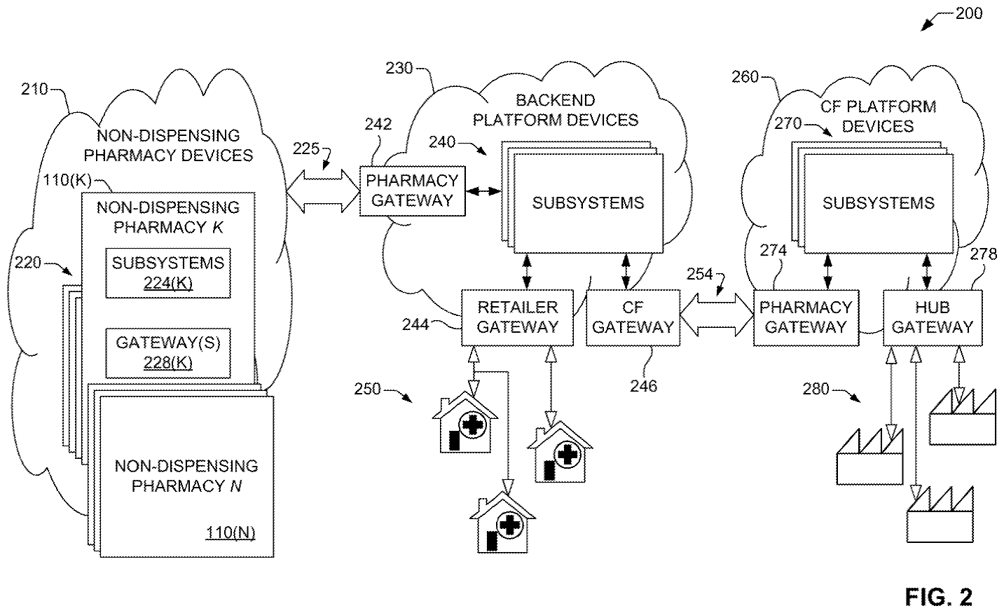

FIG. 2 illustrates an example of an architecture of the operating environment shown in FIG. 1, in accordance with one or more embodiments of this disclosure. As is illustrated in FIG. 2, each non-dispensing pharmacy 110(J), with J=1, 2, . . . , K, . . . N, can be a embodied in a group of devices within a network of devices 210. The network of devices 210 can be referred to as non-dispensing pharmacy devices 210 and includes computing devices. The group of devices includes computing resources that can provide, individually or collectively, processing functionality, storage functionality, and network connectivity functionality. The group of devices can thus constitute computing nodes, storage nodes, and gateway nodes allocated to the non-dispensing pharmacy 110(J) by a cloud computing service provider, for example. The computing nodes and storage nodes can form subsystems 224(K). The gateway nodes can form one or multiple gateways 228(K). In light of its computer-implemented aspects, each non-dispensing pharmacy 110(J) (J=1, 2, . . . , K, . . . N) can be referred to as a virtual pharmacy.

The dispensing pharmacy 120 (FIG. 1) also can be embodied in, or can include, backend platform devices 230. The backend platform devices 230 include computing resources that can provide, individually or collectively, processing functionality, storage functionality, and network connectivity functionality. As least some the backend platform devices 230 can thus constitute computing nodes, storage nodes, and gateway nodes. The computing nodes and storage nodes can constitute subsystems 240. The gateway nodes can form various types of gateways. More specifically, the backend platform devices 230 include a pharmacy gateway 242. Each non-dispensing pharmacy 110(J) can be functionally coupled to the pharmacy gateway 242 by means of a communication network 225 (a wireless network, a wireline network, or a combination of both).

A group of the subsystems 240 can form a digital prescription marketplace. The digital prescription marketplace can be associated with each retail pharmacy in a pharmacy network 250 of retail pharmacies. The pharmacy network 250 is represented with a group of three retail pharmacies simply for the purpose of illustration. The pharmacy network 250 can include fewer or more than three retailer pharmacies.

The backend platform devices 230 also include one or more gateway devices (referred to as retailer gateway 244). The retailer gateway 244 can functionally couple one or a combination of the subsystems 240 to a retailer pharmacy in the pharmacy network 250. One or several communication networks (wireless networks, wireline networks, or a combination of both; represented by open-head arrows) can connect a retailer pharmacy to the retailer gateway 244.

As also mentioned, retail pharmacies within the dispensing pharmacy platform 120 can leverage the CF platform 130 (FIG. 1). Accordingly, the backend platform devices 230 can further include gateway devices 246 (referred to as CF gateway 246) that functionally couple a retail pharmacy to the CF platform 130. Specifically, the CF platform 130 can comprises CF platform devices 260 including one or more gateway devices 274 (referred to as pharmacy gateway 274). The pharmacy gateway 274 can be functionally coupled to the CF gateway 246 by means of a communication network 254 (a wireless network, a wireline network, or a combination of both).

The CF platform devices 260 also include gateway devices 278 (referred to as CF gateway 246) that functionally couple one or a combination of the subsystems 270 to a distribution network 280 of distribution hubs. The distribution network 280 is represented with a group of three distribution hubs simply for the purpose of illustration. The distribution network 280 can include fewer or more than three retailer pharmacies.

Two types of pathways are available to the patient 150 (FIG. 1) to obtain medications. One type of pathway involves the dispensing pharmacy platform 120 as a fulfillment pharmacy for a prescription for one or multiple medications. The prescription also can be referred to as script. Another type of pathway involves a non-dispensing pharmacy of the group of dispensing pharmacies 110(1) to 110(N) as the fulfillment pharmacy for such a prescription. The prescription can be either a new prescription for the medication(s) or a refill prescription for a particular medication of the medication(s).

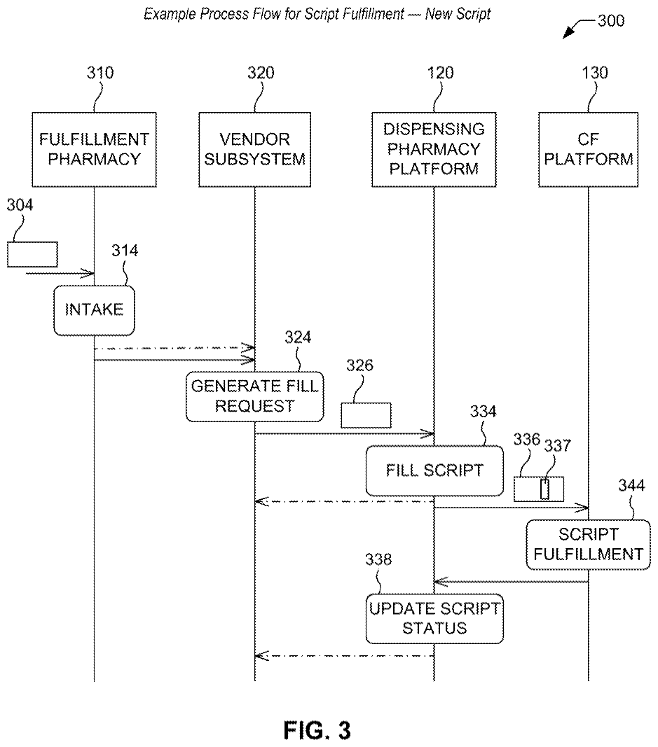

FIG. 3 illustrates an example of a process flow 300 for fulfillment of a prescription 304, in accordance with one or more embodiments of this disclosure. The prescription 304 is a new prescription for at least one medication. In some embodiments, the prescription 304 can include a prescription number or another type of prescription identifier, and also can include a dosage for at least one medication.

Regardless of the type of pathway used to obtain the at least one medication, a fulfillment pharmacy 310 receives the prescription 304. The fulfillment pharmacy 310 can be embodied in a non-dispensing pharmacy (e.g., non-dispensing pharmacy K 110(K) or the dispensing pharmacy platform 120. A source device (not depicted in FIG. 3) can provide the prescription 304. In some cases, the prescription 304 can be electronically prescribed. Accordingly, the source device can format the prescription 304 according to a communication protocol for electronic delivery, such as SCRIPT.

In response to receiving the prescription 304, one or a combination of subsystems of the fulfillment pharmacy 310 can perform an intake process 314. As part of the intake process 314, such subsystem(s) can embed a unique NPI number or a NCPDP number, or both, in a prescription data defining the prescription 304. The unique NPI number and unique NCPDP number correspond to either the non-dispensing pharmacy or the dispensing pharmacy platform 120, depending on which one of those embodies the fulfillment pharmacy 311). The intake process 314 also can include sending a notification (represented by a dash-dotted arrow in FIG. 3) to a vendor subsystem 320. The notification conveys that the prescription 304 is available for a pharmacy program (a discount card, for example). The pharmacy program may be provided by a marketplace vendor or a cohort vendor. The marketplace vendor can correspond to a retail pharmacy included in the pharmacy network 250 (FIG. 2). The cohort vendor can administer the non-dispensing pharmacy. The vendor subsystem 320 can include a tracking module that can receive the notification and can retain a record thereof.

In addition, also as part of the intake process 314, the subsystem(s) performing the intake process 314 can send a prescription fill inquiry to the vendor subsystem 320. In response to receiving the prescription fill inquiry, the vendor subsystem 320 can perform a process 324 to generate a prescription fill request. The process 324 permits completing patient intake (or onboarding) into the pharmacy program that may be provided by the marketplace vendor or the cohort vendor. The process 324 also permits obtaining data for the prescription 304 to be filled at the dispensing pharmacy platform 120. Such data can include, for example, a processor identification number (or BIN), a processor control number (PCN), a group number, or a combination thereof, pertaining to the pharmacy program provided by the marketplace vendor or the cohort vendor.

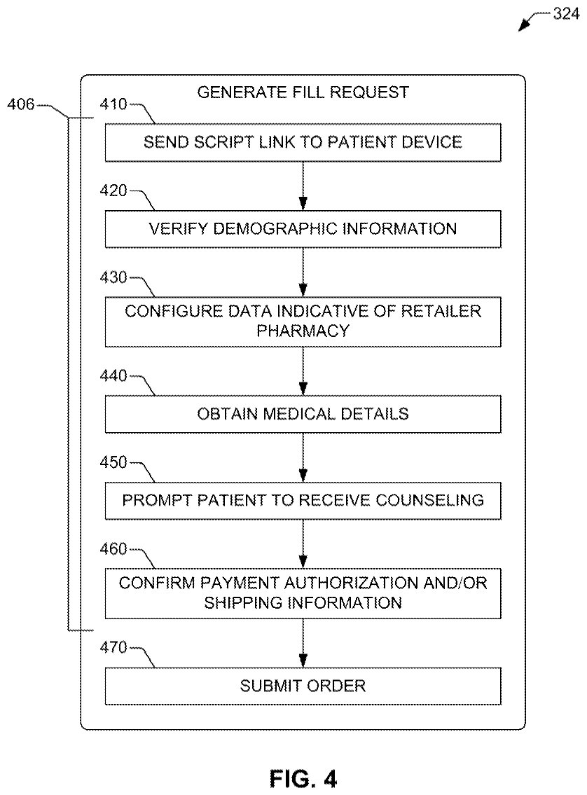

An example of the process 324 is illustrated in FIG. 4. As mentioned, the process 324 includes an onboarding stage 406 that includes multiple operations directed to acquiring information corresponding to a patient (e.g., patient 150 (FIG. 1)). Simply for purposes of illustration, the multiple operations can be grouped in blocks, each performed by the vendor subsystem 320. Specifically, at block 410, the vendor subsystem 320 can send a prescription link to a patient device. In some cases, the prescription link can be sent via an electronic message (an email, a SMS message, a MMS, an iMessage, or similar, for example). At block 420, the vendor subsystem 320 can verify demographic information corresponding to the patient. At block 430, the vendor subsystem 320 can configure data indicative of a retailer pharmacy. At block 440, the vendor subsystem 320 can obtain medical details. In some embodiments, the vendor subsystem 320 can cause the patient device to present a sequence of user interfaces (UIs) including UI elements (selectable or otherwise) that prompt input of the medical details.

At block 450, the vendor subsystem 320 can prompt input of a selection indicating either acceptance or rejection of counseling in connection with one or more particular medications included in the prescription 304. The vendor subsystem 320 can create a document, for example, including the selection and, when counseling is accepted, details conveying the counseling that has been provided. At block 460, the vendor subsystem 320 can confirm payment authorization or shipping information, or both. The shipping information includes an address where the prescribed medication(s) can be delivered. The shipping information also can include delivery instructions for a carrier delivering the prescribed medication(s).

At block 470, the vendor subsystem 320 can generate an order message. The vendor subsystem 320 also can send the message to an external computing device. In some cases, rather than sending the prescription fill request, the vendor subsystem 320 can make the message available to the external computing device. The message can be made available via an application programming interface (API) or another types of software library.

With further reference to FIG. 3, the prescription fill request that has been generated can be embodied in, or can constitute, a request message 326 formatted according to a defined communication protocol. The defined communication protocol can have messaging structure that is sanctioned and suitably configured by both the vendor subsystem 320 and one or multiple subsystems of the dispensing pharmacy platform 120. The vendor subsystem 320 can send the request message 326 to the dispensing pharmacy platform 120. As mentioned, the dispensing pharmacy platform 120 has the licensure to dispense medications. One or a combination of subsystems of the dispensing pharmacy platform 120 can perform a prescription fill process 334 to fill the prescription 304. Performing the prescription fill process 334 can include generating an order message based on the prescription fill request. The order message can have a messaging structure in accordance with a defined communication protocol. In some embodiments, the order message can be embodied in a National Council for Prescription Drug Programs (NCPDP) message.

The order message can include a custom data field to map the prescription fill for a patient (e.g., patient 150 (FIG. 1)) to the vendor subsystem 320. The custom data field can be referred to as a “cohort identifier field.” As such, data contained in the cohort identifier field can be used as a program identifier for the vendor subsystem (e.g., a cohort subsystem) that corresponds to the prescription 304 and provides a pharmacy program. Accordingly, the subsystem(s) performing the prescription fill process 334 can embed the data into the cohort identifier field. Such subsystems(s) can include a generation module that can embed the data into the cohort identifier field. In some cases, the data being embedded can include a first identifier comprising a processor identification number (or BIN) corresponding to the vendor subsystem 320. In other cases, the data being embedded can include the first identifier and a second identifier, where the second identifier can include the PCN corresponding to the vendor subsystem 320. In yet other cases, the data being embedded can include one or both of the first identifier or the second identifier in addition to a group number associated with the pharmacy program. As part of the prescription fill process 334, the subsystem(s) performing the prescription fill process 334 can complete the generation of the order message after embedding such data into the cohort identifier. By embedding BIN. PCN, and/or group number into the cohort identifier field, the subsystem(s) performing the prescription fill process 334 can correctly perform other processes that may be involved in the generation of the order message, based on NCPDP standards. Those other processes can include adjudication of a claim, for example.

The disclosure is not limited to embedding two or more of BIN, PCN, or group number into the cohort identifier field. In some embodiments, at least one of the subsystem(s) performing the prescription fill process 334 can embed particular data into the cohort identifier field, where the particular data can map the prescription 304 for a patient to the vendor subsystem 320 (e.g., a cohort subsystem). The particular data can identify a unique association between the prescription 304 and the vendor subsystem 320. The particular data can define an index that uniquely identifies the vendor subsystem 320 (such as a cohort subsystem) in some cases. As an example, the index can be embodied in a unique code (e.g., numeric or alphanumeric) assigned to the vendor subsystem 320. For instance, the code could be generated by applying a hash function to two or more of BIN for a cohort. PCN for the cohort, or a group number pertaining to a pharmacy program provided by the cohort. As another example, the index can server as a key to a table entry that identifies the cohort.

Regardless of the form of data embedded in the cohort identifier field, because the order message includes the cohort identifier field mapping a prescription for a patient and a cohort, embodiments of this disclosure can avoid or otherwise solve the issue of delineating patients and their prescriptions between cohorts associated with respective non-dispensing pharmacies. Indeed, by relying on the cohort identifier field that issue can be avoided or otherwise solved without convoluted, risky data processing or protected health information (PI-II) processing, or both, with the ensuing efficient utilization of computing resources during the fulfillment of the prescription 304. Computing resources can include network bandwidth (uplink and/or downlink), processing unit time, memory capacity (as measured by available storage in memory devices), or the similar resources available to operating environment 100 (FIG. 1) and/or other subsystems described herein. Processing unit time can be quantified by processor cycles and can include, for example, central processing unit (CPU) time, graphics processing unit (GPU) time, tensor processing unit (TPU), or time utilized by a combination of such processing units during and/or after the fulfillment of the prescription 304. The efficient utilization of those resources can mitigate network congestion and/or other types of blockade of access to computing resources available to the various subsystem described herein.

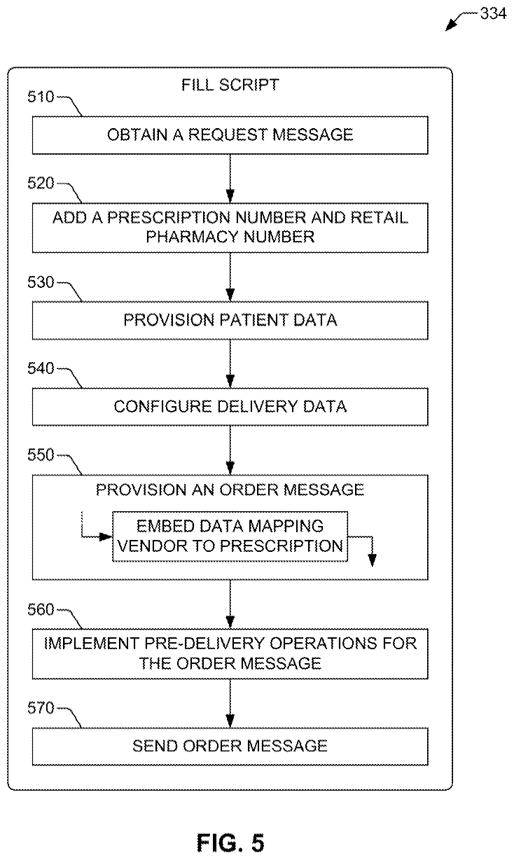

An example of the prescription fill process 334 is shown in FIG. 5. Simply for purposes of illustration, the multiple operations included in the prescription fill process 334 can be grouped in blocks, each performed by at least one of the subsystem(s) of the dispensing pharmacy platform 120. At block 510, the at least one of the subsystem(s) can obtain a request message (e.g., the request message 326). Obtaining the request message can include receiving the request message, and assigning ownership data within the workflow corresponding to the prescription fill process 334, for example. At block 520, the at least one of the subsystem(s) can add a prescription number and a retail pharmacy number. In some cases, the retail pharmacy number can be a NPI. In other cases, the at least one of the subsystem(s) can generate a unique number corresponding to the retail pharmacy number. In some embodiments, instead of adding a prescription number, the at least one of the subsystem(s) can add another type of prescription identifier, such as an alphanumeric code. The prescription identifier (numeric or otherwise) is associated with a patient and one or more medications identified in the prescription 304.

At block 530, the at least one of the subsystem(s) can provision patient data. Provisioning the patient data can include accessing a page (via a hyperlink corresponding to the patient, for example) that can receive various types of input information. Thus, provisioning the patient data also can include receiving first data identifying allergies for the patient, second data identifying medical conditions of the patient, and/or third data identifying shipping preferences (e.g., an address, special instructions, or the like). In addition, provisioning the patient data can include embedding data identifying a pharmacy program (such as a prescription discount plan) into the cohort identifier field. In some embodiments, the cohort identifier field also can be configured to include data identifying a third-party payer. Further, provisioning the patient data can include retaining the patient data into a memory device.

At block 540, the at least one of the subsystem(s) can configure delivery data. For example, the delivery data can be configured by configuring a delivery attribute to have a value equal to “Mail,” and by further configuring a second delivery attribute to a value indicative of a shipping address having been verified.

At block 550, the at least one of the subsystem(s) can provision an order message. The order message is responsive to the request message (e.g., request message 326). Provisioning the order message can include creating a data structure embodying the order message. The data structure can include the prescription number (or another type of prescription identifier), retail pharmacy number, the patient data, and the first and second delivery attribute. Provisioning the order message also can include embedding particular data into the cohort identifier field, the particular data mapping a vendor (such as a cohort vendor) to the prescription identified by the prescription number (or the other type of prescription identifier). As mentioned, the particular data being embedded can include a first identifier comprising a processor identification number (or BIN) corresponding to the vendor (e.g., a cohort subsystem). In other cases, the particular data being embedded can include the first identifier and a second identifier, where the second identifier can include the PCN corresponding to the vendor. In yet other cases, the particular data being embedded can include one or both of the first identifier or the second identifier in addition to a group number associated with a pharmacy program. As further mentioned, the particular data can be formatted in many other ways, each identifying a unique association between the prescription associated with the prescription number (or prescription identifier) and the vendor.

In addition, provisioning the order message can include completing the order message—for instance, storing in memory the data structure that embodies the order message. The memory can be embodied in one or more memory devices included in a storage subsystem within the subsystems 240.

At block 560, the at least one of the subsystem(s) can implement pre-delivery operations for the order message. Implementing the pre-delivery operations can include performing a drug utilization review (DUR) associated with the prescription 304.

After the prescription fill process 334 has been performed, the dispensing pharmacy platform 120 can send the order message to the CF platform 130. Such an order message, which has been generated as a result of performing the prescription fill process 334, is represented by a block 336 (referred to as order message 336) in FIG. 3. Additionally, the cohort identifier field that is embedded into the order message generated as a result of performing the prescription fill process 334 is represented by a block 337 within the order message 336. In some embodiments, the order message that has been generated can be sent to the CF platform 130 as part of performing the prescription fill process 334, as is shown at block 570 in FIG. 5. The subsystem(s) can send the order message to the CF platform 130. In some cases, the order message can be sent via the CF gateway 246 (FIG. 2) to the pharmacy gateway 274 (FIG. 2).

The order message can cause the CF platform 130 to dispense one or more medications pertaining to the prescription 304. That is, in response to receiving the order message, one or a combination of subsystems of the CF platform 130 can perform a script fulfillment process 344. The script fulfillment process 344 includes determining that the medication(s) are in stock and dispensing the medication(s). The script fulfillment process 344 also includes shipping the medication(s) to an address of the patient corresponding to the prescription 304.

A subsystem of the CF platform 130 can send an indication that the prescription 304 has been fulfilled. The indication can identify the order message corresponding to the prescription 304. In response to receiving that indication, a subsystem of the dispensing pharmacy platform 120 can perform a process 338 to update prescription status for the prescription 304. As part of the process 338, the subsystem can update a record indicative of the prescription 304 having been shipped from the CF platform 130. Updating the record can include creating the record or modifying the record to indicate that the prescription 304 has been shipped. Also as part of the process 338, the subsystem of the dispensing pharmacy platform 120 can send a notification (represented by a dash-dotted arrow in FIG. 3) to the vendor subsystem 320. The notification can indicate that at least one medication pertaining to the prescription 304 has been dispensed and/or shipped. In some cases, the notification can include shipping data and/or tracking data corresponding to the prescription 304. The tracking module included in the vendor subsystem 320 can receive the notification and can retain a record thereof.

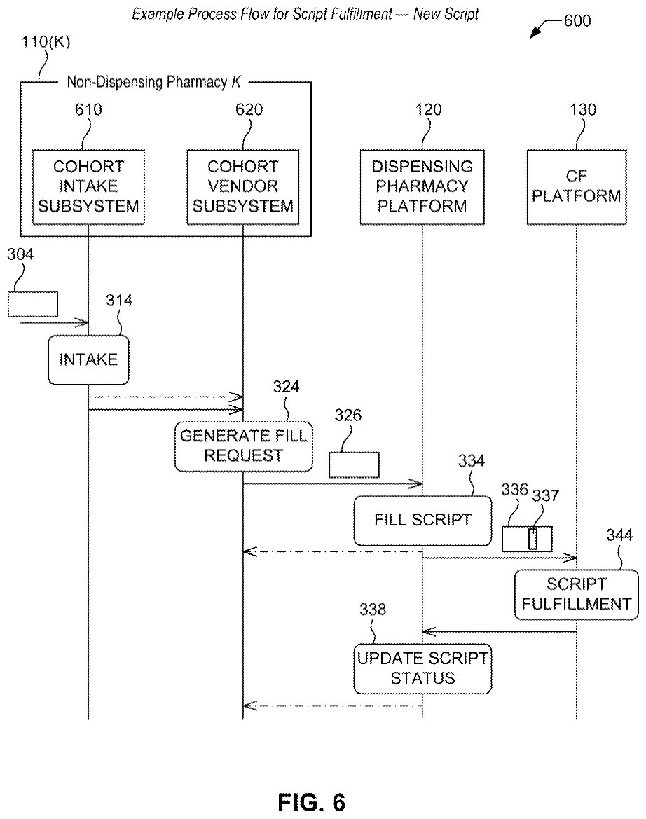

As mentioned, the fulfillment pharmacy 310 can be embodied in a non-dispensing pharmacy in accordance with aspects described of this disclosure. FIG. 6 illustrates an example of a process flow 600 for fulfillment of the prescription 304 via the non-dispensing pharmacy K 110(K). A cohort intake subsystem 610 receives the prescription 304 from a source device (not depicted in FIG. 3), for example. In response to receiving the prescription 304, the cohort intake subsystem 610 can perform the intake process 314. As part of the intake process 314, the cohort intake subsystem 610 can embed a unique NPI number or a NCPDP number, or both, in a prescription data defining the prescription 304. The unique NPI number and unique NCPDP number correspond to the non-dispensing pharmacy K 110(K). The intake process 314 also can include sending a notification (represented by a dash-dotted arrow in FIG. 3) to a cohort vendor subsystem 620. The notification conveys that the prescription 304 is available for a pharmacy program (a discount card, for example). The pharmacy program can be provided by a cohort vendor that administers the cohort vendor subsystem 620. The cohort vendor subsystem 620 can include a tracking module that can receive the notification and can retain a record thereof.

In addition, also as part of the intake process 314, the cohort intake subsystem 610 can send a prescription fill inquiry to the cohort vendor subsystem 620. In response to receiving the prescription fill inquiry, the cohort vendor subsystem 620 can perform the process 324 to generate a prescription fill request. As mentioned, the process 324 permits obtaining data for the prescription 304 to be filled at the dispensing pharmacy platform 120. Such data can include, for example, a processor identification number, a PCN, a group number, or a combination thereof, pertaining to the pharmacy program provided by the cohort vendor that administers the cohort vendor subsystem 620.

The prescription fill request that has been generated can be embodied in, or can constitute, the request message 326. The cohort vendor subsystem 620 can send the request message 326 to the dispensing pharmacy platform 120. One or a combination of subsystems of the dispensing pharmacy platform 120 can perform the prescription fill process 334 to fill the prescription 304. Performing the prescription fill process 334 can include generating an order message based on the prescription fill request. As mentioned, the order message can include a cohort identifier field to map the prescription fill for a patient (e.g., patient 150 (FIG. 1)) to the cohort vendor subsystem 620. As such, data contained in the defined field can be used as a program identifier for the cohort vendor subsystem 620 that corresponds to the prescription 304 and provides a pharmacy program. Accordingly, the subsystem(s) performing the prescription fill process 334 can embed the data into the defined field. In some cases, the data can include the first identifier comprising a processor identification number (or BIN) corresponding to the cohort vendor subsystem 620. In other cases, the data can include the first identifier and a second identifier, where the second identifier can include the PCN corresponding to the cohort vendor subsystem 620. In yet other cases, the data being embedded can include one or both of the first identifier or the second identifier in addition to a group number associated with the pharmacy program. As part of the prescription till process 334, the subsystem(s) performing the prescription fill process 334 can complete the generation of the order message after embedding such data into the defined field. As mentioned, by including BIN, PCN, and/or group number in the cohort identifier field, the subsystem(s) performing the prescription fill process 334 can correctly perform other processes that may be involved in the generation of the order message, based on NCPDP standards. Those other processes can include adjudication of a claim, for example.

After the prescription fill process 334 has been performed, the dispensing pharmacy platform 120 can send the order message to the CF platform 130. As mentioned, such an order message and the cohort identifier field embedded therein are represented by block 336 and block 337, respectively, in FIG. 6. In some embodiments, the order message that has been generated can be sent to the CF platform 130 as part of performing the prescription fill process 334 (block 570; FIG. 5).

The order message can cause the CF platform 130 to dispense one or more medications pertaining to the prescription 304. That is, in response to receiving the order message, one or a combination of subsystems of the CF platform 130 can perform a script fulfillment process 344. The script fulfillment process 344 includes determining that the medication(s) are in stock and dispensing the medication(s). The script fulfillment process 344 also includes shipping the medication(s) to an address of the patient corresponding to the prescription 304.

A subsystem of the CF platform 130 can send an indication that the prescription 304 has been fulfilled. The indication can identify the order message corresponding to the prescription 304. As mentioned, in response to receiving such an indication, a subsystem of the dispensing pharmacy platform 120 can perform the process 338 to update prescription status for the prescription 304. As part of the process 338, the subsystem can update a record indicative of the prescription 304 having been shipped from the CF platform 130. Updating the record can include creating the record or modifying the record to indicate that the prescription 304 has been shipped. Also as part of the process 338, the subsystem of the dispensing pharmacy platform 120 can send a notification (represented by a dash-dotted arrow in FIG. 3) to the cohort vendor subsystem 620. The notification can indicate that at least one medication pertaining to the prescription 304 has been dispensed and/or shipped. In some cases, the notification can include shipping data and/or tracking data corresponding to the at least one medication that has been shipped. The tracking module included in the cohort vendor subsystem 620 can receive the notification and can retain a record thereof.

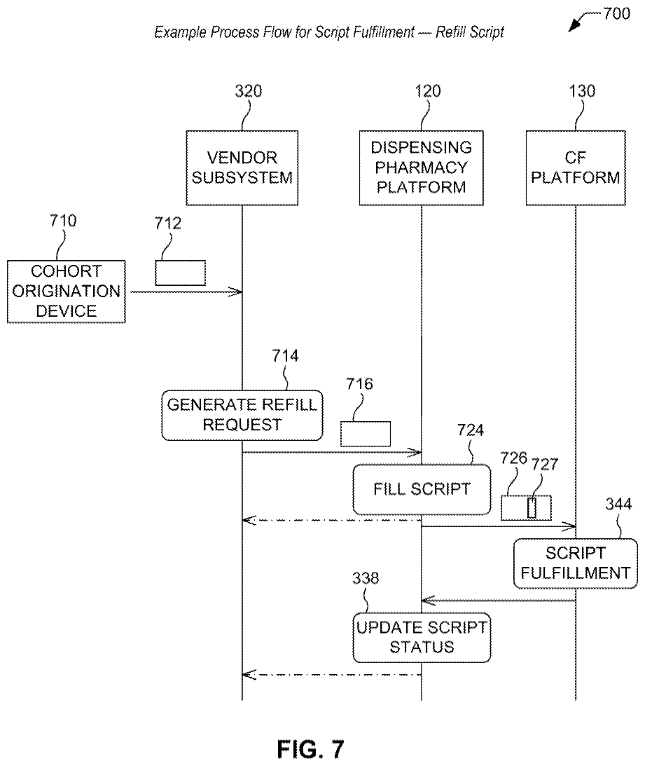

An extant prescription for the patient 150 (FIG. 1) can be refilled using either a non-dispensing pharmacy or the dispensing pharmacy platform 120, and the CF platform 130. FIG. 7 illustrates an example of a process flow 700 for fulfillment of a prescription refill, in accordance with one or more embodiments of this disclosure. A cohort origination device 710 can send a request message 712 to refill the extant prescription. In some cases, the cohort origination device 710 can be integrated into the non-dispensing pharmacy. In other cases, the cohort origination device 710 can be integrated into the dispensing pharmacy platform 120.

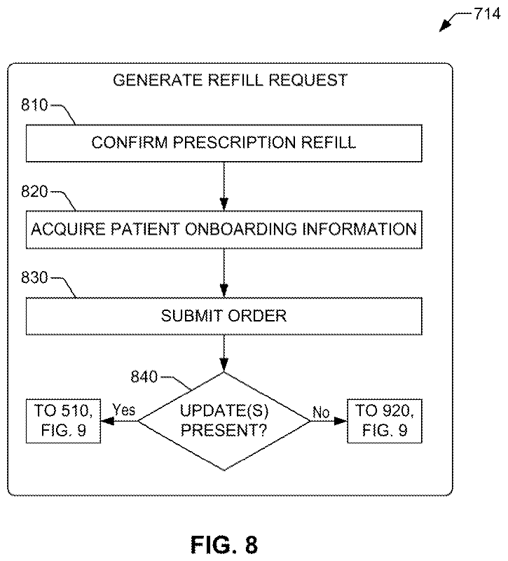

The request message 712 can be sent to the vendor subsystem 320. In response to receiving the request message 712, the vendor subsystem 320 can perform a process 714 to generate a refill request. An example of the process 714 is illustrated in FIG. 8. As part of the process 714, the vendor subsystem 320 can receive input data indicative of confirmation that a refill is desired. To the end, as is shown in FIG. 8, vendor subsystem 320 can confirm prescription refill at block 810.

Also as part of the process 714, the vendor subsystem 320 can acquire various types of current patient information. For example, the current patient information can include current clinical information, current shipping information, current demographic information, and current payment information or validation of extant payment information. The vendor subsystem 320 can acquire patient onboarding information at block 820, as is shown in FIG. 8.

The vendor subsystem 320 can generate a prescription refill request after the refill is confirmed and the current patient information is acquired. The prescription refill request can be embodied in, or can constitute, a prescription refill message 716. The vendor subsystem 320 can generate the prescription refill request at block 830, as is shown in FIG. 8.

The current patient information can include changes relative to past patient information acquired as part of the original fulfillment of the extant prescription. Accordingly, prior to sending the prescription refill message 716, also as part of the process 714, the vendor subsystem 320 can determine if an update is present in the current patient information relative to the past patient information. As is shown in FIG. 8, at block 840, the vendor subsystem 320 can determine if such an update is present. Presence or absence of the update can determine the stage in which the prescription fill process 724 is initiated. In cases where updates are absent, the vendor subsystem 320 can send the prescription refill message 716 to a terminal stage of the prescription fill process 724. That terminal stage can include, for example, performing a DUR. Because updates are absent, after implementing the terminal stage as needed, the dispensing pharmacy platform 120 can generate a current order message for the refill request by replicating an extant order message for fulfillment of the original prescription. As such, the current order message also includes the cohort identifier field that maps the prescription refill to the vendor subsystem 320 (the cohort vendor subsystem corresponding to a non-dispensing pharmacy, for example).

In the alternative, in cases where updates are present in the current patient information, the vendor subsystem 320 can send the prescription refill message 716 to an initial stage of the prescription fill process 724. Hence, one or a combination of subsystems of the dispensing pharmacy platform 120 can generate a current order message that includes updated data. In some cases, the current order message can contain an updated cohort identifier field that associates the prescription refill to an updated vendor subsystem. Accordingly, the subsystem(s) performing the prescription fill process 724 can embed updated data into the defined field. The updated data can include, for example, a first identifier comprising a processor identification number (or BIN) corresponding to the updated vendor subsystem. In addition, or in other cases, the data can include the first identifier and a second identifier, where the second identifier can include the PCN corresponding to the updated vendor subsystem. As part of the prescription fill process 724, the subsystem(s) performing the prescription fill process 724 can complete the generation of the current order message after embedding such data into the defined field.

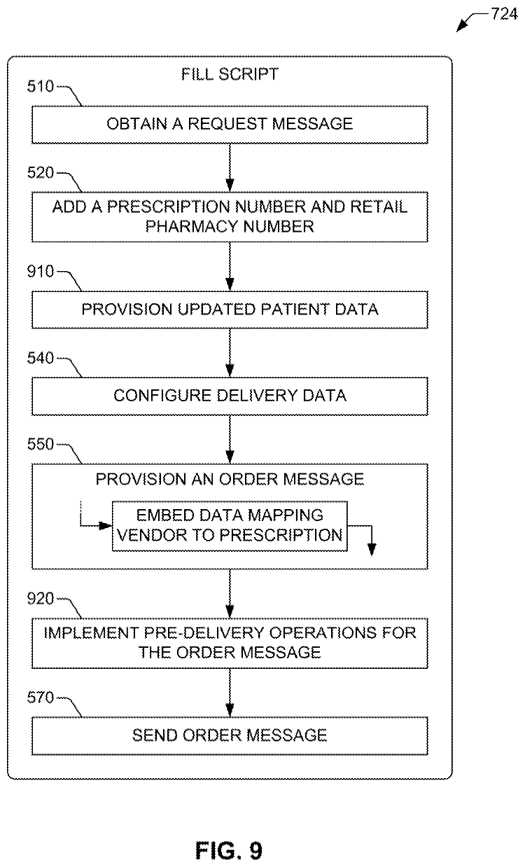

An example of the prescription fill process 724 is shown in FIG. 9. Simply for purposes of illustration, the multiple operations included in the prescription fill process 724 can be grouped in blocks, each performed by at least one of the subsystem(s) of the dispensing pharmacy platform 120. The prescription fill process 724 is similar to the prescription fill process 334, except for block 910 and block 920. Such blocks can be implemented in response to a determination of presence and absence of an update, respectively.

In connection with block 910, the at least one of the subsystem(s) can provision updated patient data. Provisioning the updated patient data can include accessing a page (via a hyperlink corresponding to the patient, for example) that can receive various types of input information. Thus, provisioning the update patient data also can include receiving first updated data identifying newly developed allergies for the patient, second updated data identifying newly developed medical conditions of the patient, and/or third updated data identifying new shipping preferences (e.g., new address, new special instructions, or the like). In addition, in some cases, provisioning the updated patient data can include embedding data identifying a new pharmacy program (such as a prescription discount plan) into a custom data field. Again, the custom data field can be referred to as a cohort identifier field. In some embodiments, the cohort identifier field also can be configured to include data identifying a third-party payer. Further, provisioning the updated patient data can include retaining the patient data into a memory device.

In connection with block 920, the at least one of the subsystem(s) can implement one or multiple pre-delivery operations for the order message. Implementing the pre-delivery operation(s) can include performing a DUR associated with the prescription 304 that is to be refilled.

After the prescription fill process 724 has been performed, a subsystem of the dispensing pharmacy platform 120 can send the current order message to the CF platform 130. Such an order message, which has been generated as a result of performing the prescription fill process 724, is represented by a block 726 (referred to as order message 726) in FIG. 7. Additionally, the cohort identifier field that is embedded into the order message generated as a result of performing the prescription fill process 724 is represented by a block 727 within the order message 726. In some embodiments, the current order message that has been generated can be sent to the CF platform 130 as part of performing the prescription fill process 724 (block 570; FIG. 9).

The current order message can cause the CF platform 130 to dispense one or more medications pertaining to the prescription refill corresponding to the request message 712. That is, in response to receiving the order message, one or a combination of subsystems of the CF platform 130 can perform the prescription fulfillment process 344 using the current order message as input, for a particular medication of one or multiple medications included in an original prescription.

As is described herein, a subsystem of the CF platform 130 can send an indication that the prescription refill has been fulfilled. The indication can identify the order message corresponding to the prescription refill. In response to receiving that indication, a subsystem of the dispensing pharmacy platform 120 can perform the process 338 to update prescription status for the prescription refill. As part of the process 338, the subsystem can update a record indicative of the particular medication in the prescription refill having been shipped from the CF platform 130. Updating the record can include creating the record or modifying the record to indicate that the particular medication has been shipped. Also as part of the process 338, the subsystem of the dispensing pharmacy platform 120 can send a notification (represented by a dash-dotted arrow in FIG. 7) to the vendor subsystem 320. The notification can indicate that the particular medication corresponding to the refill request has been dispensed and/or shipped. In some cases, the notification can include shipping data and/or tracking data corresponding to the particular medication that has been shipped. The tracking module included in the vendor subsystem 320 can receive the notification and can retain a record thereof.

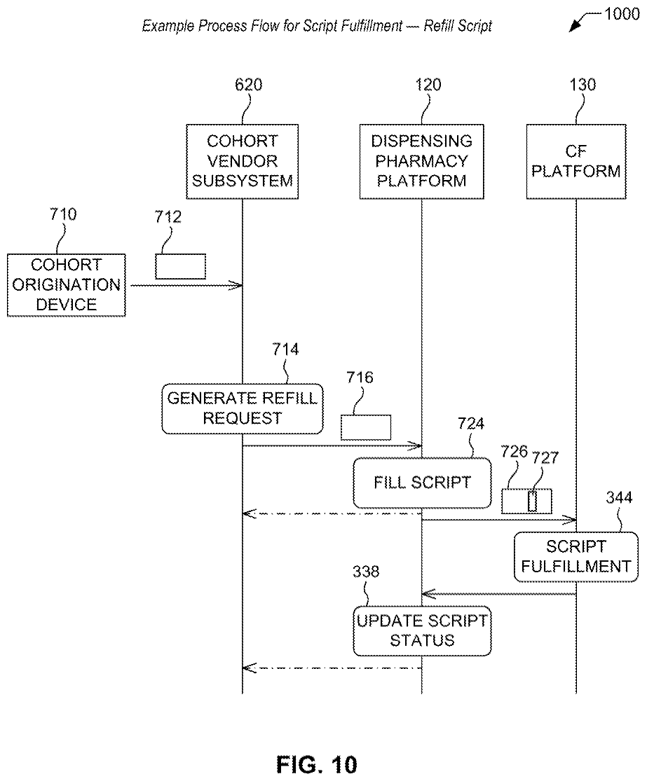

FIG. 10 illustrates an example of a process flow 1000 for fulfillment of a prescription refill, in accordance with one or more embodiments of this disclosure. Fulfillment of the prescription refill relies on the cohort vendor subsystem 620 (FIG. 6) associated with the non-dispensing pharmacy K 110(K). The cohort origination device 710 can send the request message 712 to refill an extant prescription. The cohort origination device 710 can be integrated into the non-dispensing pharmacy K 110(K). The request message 712 can be sent to the cohort vendor subsystem 620. In response to receiving the request message 712, the cohort vendor subsystem 620 can perform the process 714 to generate a refill request.

As part of the process 714, the cohort vendor subsystem 620 can receive input data indicative of confirmation that a refill is desired. Also as part of the process 714, the cohort vendor subsystem 620 can acquire various types of current patient information (e.g., current clinical information, current shipping information, current demographic information, and current payment information or validation of extant payment information). The cohort vendor subsystem 620 can generate a prescription refill request after the refill is confirmed and the current patient information is acquired. As mentioned, the prescription refill request can be embodied in, or can constitute, the prescription refill message 716.

Prior to sending the prescription refill message 716, also as part of the process 714, the cohort vendor subsystem 620 can determine if an update is present in current patient information relative to past patient information. Presence or absence of the update can determine the stage in which the prescription fill process 724 is initiated. In cases where updates are absent, the cohort vendor subsystem 620 can send the prescription refill message 716 to a terminal stage of the prescription fill process 724. That terminal stage can include, for example, performing a DUR. Because updates are absent, after implementing the terminal stage as needed, the dispensing pharmacy platform 120 can generate a current order message for the refill request by replicating an extant order message for fulfillment of the original prescription. As such, the current order message also includes the cohort identifier field that associates the prescription refill to the cohort vendor subsystem 620-namely, the cohort vendor subsystem corresponding to a non-dispensing pharmacy.

In the alternative, in cases where updates are present in the current patient information, the cohort vendor subsystem 620 can send the prescription refill message 716 to an initial stage of the prescription fill process 724. Hence, one or a combination of subsystems of the dispensing pharmacy platform 120 can generate a current order message that includes updated data. In some cases, the current order message can contain an updated cohort identifier field that associates the prescription refill to an updated cohort vendor subsystem, such as another non-dispensing pharmacy. Accordingly, the subsystem(s) performing the prescription fill process 724 can embed updated data into the updated cohort identifier field. The updated data being embedded can include, for example, a first identifier comprising a processor identification number (or BIN) corresponding to the updated cohort vendor subsystem. In addition, or in other cases, the data being embedded can include the first identifier and a second identifier, where the second identifier can include the PCN corresponding to the updated cohort vendor subsystem. Further, or in yet other cases, the updated data being embedded can include one or both of the first identifier or the second identifier in addition to a group number associated with a pharmacy program provided by the cohort vendor subsystem 620, for example. As part of the prescription fill process 724, the subsystem(s) performing the prescription fill process 724 can complete the generation of the current order message after embedding such data into the defined field.

After the prescription fill process 724 has been performed, a subsystem of the dispensing pharmacy platform 120 can send the current order message to the CF platform 130. As mentioned, such an order message and the cohort identifier field embedded therein are represented by block 726 and block 727, respectively, in FIG. 10. In some embodiments, the current order message that has been generated can be sent to the CF platform 130 as part of performing the process 724 (block 570; FIG. 9).

The current order message can cause the CF platform 130 to dispense one or more medications pertaining to the prescription refill corresponding to the request message 712. That is, in response to receiving the order message, one or a combination of subsystems of the CF platform 130 can perform the prescription fulfillment process 344 using the current order message as input, for a particular medication of one or multiple medications included in an original prescription.

As is described herein, a subsystem of the CF platform 130 can send an indication that the prescription refill has been fulfilled. The indication can identify the order message corresponding to the prescription refill. In response to receiving that indication, a subsystem of the dispensing pharmacy platform 120 can perform the process 338 to update prescription status for the prescription refill. As part of the process 338, the subsystem can update a record indicative of the particular medication in the prescription refill having been shipped from the CF platform 130. Updating the record can include creating the record or modifying the record to indicate that the particular medication has been shipped. Also as part of the process 338, the subsystem of the dispensing pharmacy platform 120 can send a notification (represented by a dash-dotted arrow in FIG. 7) to the vendor subsystem 320. The notification can indicate that the particular medication corresponding to the refill request has been dispensed and/or shipped. In some cases, the notification can include shipping data and/or tracking data corresponding to the particular medication that has been shipped. The tracking module included in the vendor subsystem 320 can receive the notification and can retain a record thereof.

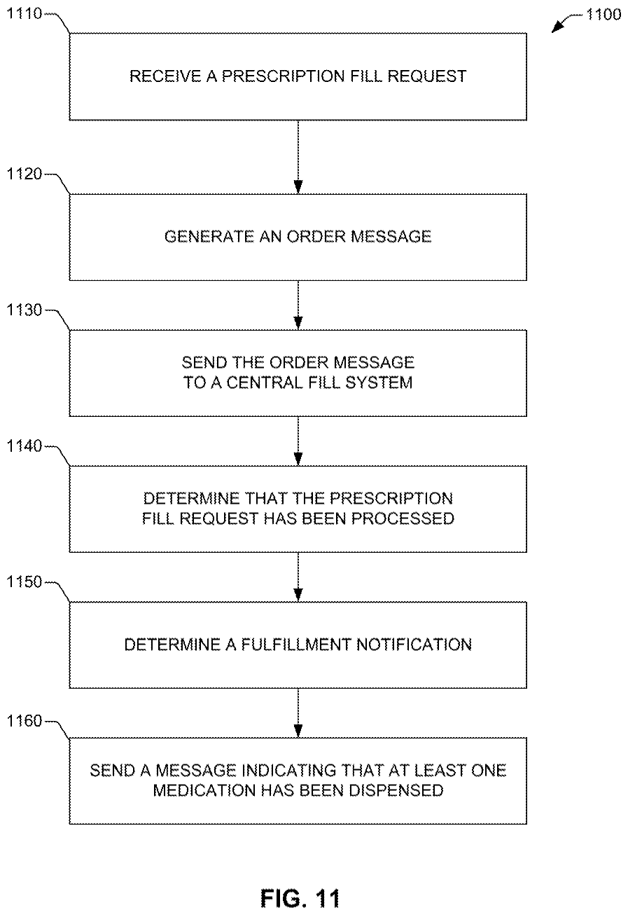

FIG. 11 illustrates an example of a method 1100 for filling a prescription, in accordance with one or more embodiments of this disclosure. A computing system can implement the example method 1100 in its entirety or in part. To that end, the computing system includes computing resources that can implement at least one of the blocks included in the example method 1100. The computing resources include, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources. For instance, the computing system can include programming interface(s); an operating system: software for configuration and or control of a virtualized environment; firmware; and similar resources.

In some embodiments, the computing system can embody, or can constitute, one or a combination of the subsystems 240 (FIG. 2). The computing system can be integrated into a dispensing pharmacy platform and, thus, can be referred to as a dispensing pharmacy system.

At block 1110, the computing system can receive a prescription fill request. In one example, the prescription fill request can be embodied in the request message 326. The prescription request can comprise a prescription identifier associated with a patient and at least one medication; a first identifier associated with the first non-dispensing pharmacy system: and a second identifier associated with the first non-dispensing pharmacy system.

At block 1120, the computing system can generate, based on the prescription fill request, an order message associated with the prescription identifier. In some embodiments, a first portion of the order message comprises the first identifier and the second identifier. Such a first portion can embody a cohort identifier field. In one example, the order message can be embodied in the order message 336.

At block 1130, the computing system can send the order message to a central fill system. The central fill system can be integrated within the CF platform 130 (FIG. 1) for example. The order message causes the central fill system to dispense the at least one medication.

At block 1140, the computing system can determine that the prescription fill request has been processed. For example, the computing system can determine that the prescription fill request has been processed based on an indication from the central fill system, That indication identifies the order message.

At block 1150, the computing system can determine a fulfillment notification. For example, the computing system can determine the fulfillment notification based on the prescription fill request having been processed. Additionally, or in the alternative, the computing system can determine the fulfillment notification based on the first portion of the order message. The fulfillment notification may indicate the prescription fill request has been processed. In some embodiments, determining the fulfillment notification can include receiving the indication that identifies the order message; retrieving an index comprising a plurality of mappings, where each mapping identifies one of multiple non-dispensing pharmacy systems; determining, based on the indication identifying the order message, and based on the first portion of the order message (e.g., a cohort identifier field), that a first mapping of the plurality of mappings is associated with the first non-dispensing pharmacy system, where the first mapping can include the first identifier and the second identifier associated with the first non-dispensing pharmacy system. Determining the fulfillment notification can include determining, based on the prescription fill request having been processed, and based on the first mapping, the fulfillment notification.

At block 1160, the computing system can send a message indicating that the at least one medication has been dispensed. For example, the computing system can send the message indicating that the at least one medication has been dispensed based on the fulfillment notification. In some embodiments, such a message can be embodied in, or can include, the notification represented by the dash-dotted arrow from the dispensing pharmacy platform 120 to the vendor subsystem 320.

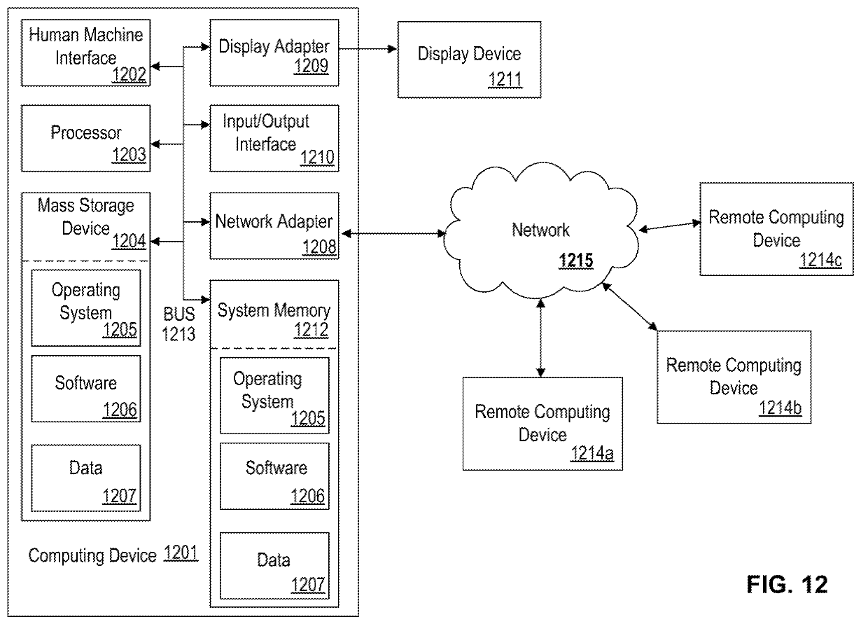

In order to provide some context, the computer-implemented method and systems of this disclosure can be implemented on the computing environment illustrated in FIG. 12 and described below. Similarly, the computer-implemented methods and systems disclosed herein can utilize one or more computing devices to perform one or more functions in one or more locations. FIG. 12 is a block diagram illustrating an example of a computing environment for performing the disclosed methods and/or implementing the disclosed systems. The operating environment shown in FIG. 12 is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment. The operating environment shown in FIG. 12 can embody at least a portion of the operating environment 200 (FIG. 2).

The computer-implemented methods and systems in accordance with this disclosure can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set-top boxes, programmable consumer electronics, network PCs, minicomputers, mainframe computers, distributed computing environments that comprise any of the above systems or devices, and the like.

The processing of the disclosed computer-implemented methods and systems can be performed by software components. The disclosed systems and computer-implemented methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and computer-implemented methods disclosed herein can be implemented via a general-purpose computing device in the form of a computing device 1201. The components of the computing device 1201 can comprise, but are not limited to, one or more processors 1203, a system memory 1212, and a system bus 1213 that couples various system components including the one or more processors 1203 to the system memory 1212. The system can utilize parallel computing.

The system bus 1213 represents one or more of several possible types of bus structures, including a memory bus or memory controller, a peripheral bus, an accelerated graphics port, or local bus using any of a variety of bus architectures. The bus 1213, and all buses specified in this description can also be implemented over a wired or wireless network connection and each of the subsystems, including the one or more processors 1203, a mass storage device 1204, an operating system 1205, software 1206, data 1207, a network adapter 1208, the system memory 1212, an Input/Output Interface 1210, a display adapter 1209, a display device 1211, and a human-machine interface 1202, can be contained within one or more remote computing devices 1214a,b,c at physically separate locations, connected through buses of this form, in effect implementing a fully distributed system.

The computing device 1201 typically comprises a variety of computer-readable media. Exemplary readable media can be any available media that is accessible by the computing device 1201 and comprises, for example and not meant to be limiting, both volatile and non-volatile media, removable and non-removable media. The system memory 1212 comprises computer readable media in the form of volatile memory, such as random access memory (RAM), and/or non-volatile memory, such as read only memory (ROM). The system memory 1212 typically contains data such as the data 1207 and/or program modules such as the operating system 1205 and the software 1206 that are immediately accessible to and/or are presently operated on by the one or more processors 1203.

In another aspect, the computing device 1201 can also comprise other removable/non-removable, volatile/non-volatile computer storage media. By way of example, FIG. 12 illustrates the mass storage device 1204 which can provide non-volatile storage of computer code, computer readable instructions, data structures, program modules, and other data for the computing device 1201. For example and not meant to be limiting, the mass storage device 1204 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 1204, including by way of example, the operating system 1205 and the software 1206. Each of the operating system 1205 and the software 1206 (or some combination thereof) can comprise elements of the programming and the software 1206. The data 1207 can also be stored on the mass storage device 1204. The data 1207 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computing device 1201 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the one or more processors 1203 via the human-machine interface 1202 that is coupled to the system bus 1213, but can be connected by other interface and bus structures, such as a parallel port, game port, an IEEE 1394 Port (also known as a Firewire port), a serial port, or a universal serial bus (USB).

In yet another aspect, the display device 1211 can also be connected to the system bus 1213 via an interface, such as the display adapter 1209. It is contemplated that the computing device 1201 can have more than one display adapter 1209 and the computing device 1201 can have more than one display device 1211. For example, the display device 1211 can be a monitor, an LCD (Liquid Crystal Display), or a projector. In addition to the display device 1211, other output peripheral devices can comprise components such as speakers (not shown) and a printer (not shown) which can be connected to the computing device 1201 via the Input/Output Interface 1210. Any operation and/or result of the methods can be output in any form to an output device. Such output can be any form of visual representation, including, but not limited to, textual, graphical, animation, audio, tactile, and the like. The display device 1211 and computing device 1201 can be part of one device, or separate devices.

The computing device 1201 can operate in a networked environment using logical connections to one or more remote computing devices 1214a,b,e. By way of example, a remote computing device can be a personal computer, portable computer, smartphone, a server, a router, a network computer, a peer device or other common network node, and so on. Logical connections between the computing device 1201 and a remote computing device 1214a,b,c can be made via a network 1215, such as a local area network (LAN) and/or a general wide area network (WAN). Such network connections can be through the network adapter 1208. The network adapter 1208 can be implemented in both wired and wireless environments. In an aspect, one or more of the remote computing devices 1214a,b,c can comprise an external engine and/or an interface to the external engine.

For purposes of illustration, application programs and other executable program components such as the operating system 1205 are illustrated herein as discrete blocks, although it is recognized that such programs and components reside at various times in different storage components of the computing device 1201, and are executed by the one or more processors 1203 of the computer. An implementation of the software 1206 can be stored on or transmitted across some form of computer-readable media. Any of the disclosed methods can be performed by computer readable instructions embodied on computer-readable media. Computer-readable media can be any available media that can be accessed by a computer. By way of example and not meant to be limiting, computer-readable media can comprise “computer storage media” and “communications media.” “Computer storage media” comprise volatile and non-volatile, removable and non-removable media implemented in any methods or technology for storage of information such as computer-readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

It is to be understood that the methods and systems described here are not limited to specific operations, processes, components, or structure described, or to the order or particular combination of such operations or components as described. It is also to be understood that the terminology used herein is for the purpose of describing exemplary embodiments only and is not intended to be restrictive or limiting.

As used herein the singular forms “a,” “an,” and “the” include both singular and plural referents unless the context clearly dictates otherwise. Values expressed as approximations, by use of antecedents such as “about” or “approximately,” shall include reasonable variations from the referenced values. If such approximate values are included with ranges, not only are the endpoints considered approximations, the magnitude of the range shall also be considered an approximation. Lists are to be considered exemplary and not restricted or limited to the elements comprising the list or to the order in which the elements have been listed unless the context clearly dictates otherwise.

Throughout the specification and claims of this disclosure, the following words have the meaning that is set forth: “comprise” and variations of the word, such as “comprising” and “comprises,” mean including but not limited to, and are not intended to exclude, for example, other additives, components, integers, or operations. “Include” and variations of the word, such as “including” are not intended to mean something that is restricted or limited to what is indicated as being included, or to exclude what is not indicated. “May” means something that is permissive but not restrictive or limiting. “Optional” or “optionally” means something that may or may not be included without changing the result or what is being described. “Prefer” and variations of the word such as “preferred” or “preferably” mean something that is exemplary and more ideal, but not required. “Such as” means something that serves simply as an example.

Operations and components described herein as being used to perform the disclosed methods and construct the disclosed systems are illustrative unless the context clearly dictates otherwise. It is to be understood that when combinations, subsets, interactions, groups, etc. of these operations and components are disclosed, that while specific reference of each various individual and collective combinations and permutation of these may not be explicitly disclosed, each is specifically contemplated and described herein, for all methods and systems. This applies to all aspects of this application including, but not limited to, operations in disclosed methods and/or the components disclosed in the systems. Thus, if there are a variety of additional operations that can be performed or components that can be added, it is understood that each of these additional operations can be performed and components added with any specific embodiment or combination of embodiments of the disclosed systems and methods.

Embodiments of this disclosure may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices, whether internal, networked, or cloud-based.

Embodiments of this disclosure have been described with reference to diagrams, flowcharts, and other illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operations to be performed on the computer or other programmable apparatus to produce a computer-implemented process such that the instructions that execute on the computer or other programmable apparatus provide operations for implementing the functions specified in the flowchart block or blocks.

Accordingly, blocks of the block diagrams and flowchart illustrations support combinations of means for performing the specified functions, combinations of operations for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or operations, or combinations of special purpose hardware and computer instructions.

The methods and systems can employ Artificial Intelligence techniques such as machine learning and iterative learning. Examples of such techniques include, but are not limited to, expert systems, case-based reasoning, Bayesian networks, behavior-based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

While the computer-implemented methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its operations be performed in a specific order. Accordingly, where a method claim does not actually recite an order to be followed by its operations or it is not otherwise specifically stated in the claims or descriptions that the operations are to be limited to a specific order, it is in no way intended that an order be inferred, in any respect. This holds for any possible non-express basis for interpretation, including: matters of logic with respect to arrangement of operations or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
receiving, by a dispensing pharmacy system, from a first non-dispensing pharmacy system, a prescription fill request comprising:
a prescription identifier associated with a patient and at least one medication,
a first identifier associated with the first non-dispensing pharmacy system, and
a second identifier associated with the first non-dispensing pharmacy system;
generating, based on the prescription fill request, an order message associated with the prescription identifier, wherein a first portion of the order message comprises the first identifier and the second identifier;
sending, to a central fill system, the order message, wherein the order message causes the central fill system to dispense the at least one medication;
determining, based on an indication from the central fill system, that the prescription fill request has been processed, wherein the indication identifies the order message;
determining, based on the prescription fill request having been processed, and based on the first portion of the order message, a fulfillment notification indicating the prescription fill request has been processed; and
sending, based on the fulfillment notification, a message indicating that the at least one medication has been dispensed.

2. The method of claim 1, wherein determining the fulfillment notification comprises:
receiving, from the central fill system, the indication;
retrieving an index comprising a plurality of mappings, wherein each mapping identifies one of a plurality of non-dispensing pharmacy systems;
determining, based on the indication identifying the order message, and based on the first portion of the order message, that a first mapping of the plurality of mappings is associated with the first non-dispensing pharmacy system, wherein the first mapping comprises the first identifier and the second identifier associated with the first non-dispensing pharmacy system; and
determining, based on the prescription fill request having been processed, and based on the first mapping, the fulfillment notification.

3. The method of claim 1, wherein the indication identifies the order message and an address associated with the patient, and wherein the message indicates that the at least one medication was dispensed via a shipment associated with the address.

4. The method of claim 1, wherein the first identifier comprises a first processor identification number.

5. The method of claim 1, wherein the second identifier comprises a processor control number.

6. The method of claim 1, wherein the order message comprises a National Council for Prescription Drug Programs (NCPDP) message, and wherein the first portion of the order message comprises at least one field of the NCPDP message configured to identify a third-party payer.

7. The method of claim 1, further comprising:
receiving, by the dispensing pharmacy system, from another dispensing pharmacy system or a provider system, a prescription comprising the prescription identifier and a dosage for the at least one medication;
sending, to the first non-dispensing pharmacy system, a prescription fill inquiry associated with the prescription; and
receiving, from the first non-dispensing pharmacy system, the prescription fill request.

8. An apparatus comprising:
one or more processors; and
memory storing computer-executable instructions that, when executed by the one or more processors, cause the apparatus to:
receive, from a first non-dispensing pharmacy system, a prescription till request comprising:
a prescription identifier associated with a patient and at least one medication,
a first identifier associated with the first non-dispensing pharmacy system, and
a second identifier associated with the first non-dispensing pharmacy system;
generate, based on the prescription fill request, an order message associated with the prescription identifier, wherein a first portion of the order message comprises the first identifier and the second identifier;
send, to a central fill system, the order message, wherein the order message causes the central fill system to dispense the at least one medication;
determine, based on an indication from the central fill system, that the prescription fill request has been processed, wherein the indication identifies the order message;
determine, based on the prescription fill request having been processed, and based on the first portion of the order message, a fulfillment notification indicating the prescription fill request has been processed; and
send, based on the fulfillment notification, a message indicating that the at least one medication was dispensed.

9. The apparatus of claim 8, wherein the computer-executable instructions that cause the apparatus to determine the fulfillment notification further cause the apparatus to:
receive, from the central fill system, the indication:
retrieve an index comprising a plurality of mappings, wherein each mapping identifies one of a plurality of non-dispensing pharmacy systems;
determine, based on the indication identifying the order message, and based on the first portion of the order message, that a first mapping of the plurality of mappings is associated with the first non-dispensing pharmacy system, wherein the first mapping comprises the first identifier and the second identifier associated with the first non-dispensing pharmacy system; and
determine, based on the prescription fill request having been processed, and based on the first mapping, the fulfillment notification.

10. The apparatus of claim 8, wherein the indication identifies the order message and an address associated with the patient, and wherein the message indicates that the at least one medication was dispensed via a shipment associated with the address.

11. The apparatus of claim 8, wherein the first identifier comprises a first processor identification number.

12. The apparatus of claim 8, wherein the second identifier comprises a processor control number.

13. The apparatus of claim 8, wherein the order message comprises a National Council for Prescription Drug Programs (NCPDP) message, and wherein the first portion of the order message comprises at least one field of the NCPDP message configured to identify a third-party payer.

14. The apparatus of claim 8, wherein the computer-executable instructions further cause the apparatus to:
receive, from another dispensing pharmacy system or a provider system, a prescription comprising the prescription identifier and a dosage for the at least one medication;
send, to the first non-dispensing pharmacy system, a prescription fill inquiry associated with the prescription; and
receive, from the first non-dispensing pharmacy system, the prescription fill request.

15. A non-transitory computer-readable storage medium comprising processor-executable instructions that, when executed by one or more processors of a computing device, cause the computing device to:
receive, from a first non-dispensing pharmacy system, a prescription fill request comprising:
a prescription identifier associated with a patient and at least one medication,
a first identifier associated with the first non-dispensing pharmacy system, and
a second identifier associated with the first non-dispensing pharmacy system;
generate, based on the prescription fill request, an order message associated with the prescription identifier, wherein a first portion of the order message comprises the first identifier and the second identifier;
send, to a central fill system, the order message, wherein the order message causes the central fill system to dispense the at least one medication:
determine, based on an indication from the central fill system, that the prescription fill request has been processed, wherein the indication identifies the order message;
determine, based on the prescription fill request having been processed, and based on the first portion of the order message, a fulfillment notification indicating the prescription fill request has been processed, and
send, based on the fulfillment notification, a message indicating that the at least one medication was dispensed.

16. The non-transitory computer-readable storage medium of claim 15, wherein the processor-executable instructions that cause the computing device to determine the fulfillment notification further cause the computing device to:
receive, from the central till system, the indication;
retrieve an index comprising a plurality of mappings, wherein each mapping identifies one of a plurality of non-dispensing pharmacy systems;
determine, based on the indication identifying the order message, and based on the first portion of the order message, that a first mapping of the plurality of mappings is associated with the first non-dispensing pharmacy system, wherein the first mapping comprises the first identifier and the second identifier associated with the first non-dispensing pharmacy system; and
determine, based on the prescription fill request having been processed, and based on the first mapping, the fulfillment notification.

17. The non-transitory computer-readable storage medium of claim 15, wherein the indication identifies the order message and an address associated with the patient, and wherein the message indicates that the at least one medication was dispensed via a shipment associated with the address.

18. The non-transitory computer-readable storage medium of claim 15, wherein the first identifier comprises a first processor identification number, and wherein the second identifier comprises a processor control number.

19. The non-transitory computer-readable storage medium of claim 15, wherein the order message comprises a National Council for Prescription Drug Programs (NCPDP) message, and wherein the first portion of the order message comprises at least one field of the NCPDP message configured to identifier a third-party payer.

20. The non-transitory computer-readable storage medium of claim 15, wherein the processor-executable instructions further cause the apparatus to:
receive, from another dispensing pharmacy system or a provider system, a prescription comprising the prescription identifier and a dosage for the at least one medication;
send, to the first non-dispensing pharmacy system, a prescription fill inquiry associated with the prescription; and
receive, from the first non-dispensing pharmacy system, the prescription fill request.
